(19) United States
(12) Patent Application Publication
(10) Pub. No.: US 2016/0263388 A1
(43) Pub. Date: Sep. 15, 2016

(54) RADIOFREQUENCY TREATMENT PROBE FOR TREATING FECAL INCONTINENCE AND ASSOCIATED SYSTEMS AND METHODS

(71) Applicant: ThermiGen, LLC, Southlake, TX (US)

(72) Inventors: Red Alinsod, Laguna Beach, CA (US); Paul R. Herchman, JR., Grapvevine, TX (US); Kevin D. O'Brien, Coppell, TX (US)

(21) Appl. No.: 14/844,092

(22) Filed: Sep. 3, 2015

Related U.S. Application Data
(63) Continuation of application No. 14/641,435, filed on Mar. 9, 2015.

(51) Int. Cl. A61N 1/40; A61N 1/08; A61N 1/05

(52) U.S. Cl. CPC A61N 1/403; A61N 1/0512; A61N 1/08

(57) ABSTRACT

The present invention is related to a treatment probe and method for treating fecal incontinence. The treatment probe may include a straight, rounded treatment tip designed to accommodate rectal anatomy, an electrode assembly coupled to the treatment tip, wherein the electrode coupled to the treatment tip is configured to transfer radiofrequency energy to specific rectal structures, a temperature measuring feature coupled to the electrode assembly, wherein the temperature measuring feature coupled to the electrode assembly is configured to monitor and regulate electrode and skin temperature, a radiofrequency handle configured to connect to the treatment tip, a connector configured to connect the radiofrequency handle to a radiofrequency generator, and a protective apparatus configured to protect the radiofrequency handle.

### RELATED APPLICATIONS

This application is a continuation-in-part and claims the benefit under 35 U.S.C. §120 of U.S. patent application Ser. No. 14/641,435, filed on Mar. 9, 2015.

### FIELD OF THE INVENTION

The present invention relates to systems and methods for treating fecal incontinence and, more specifically, to treating fecal incontinence by stimulating rectal nerves and muscle tissue.

### BACKGROUND

Fecal incontinence has a significant effect on the quality of life of the affected person. Fecal incontinence is the involuntary loss of feces and can impact area of life.

There are two main forms of fecal incontinence depending on the mechanism of incontinence. The first is urge incontinence where the person has a desire to defecate but incontinence occurs despite the person's efforts to retain stool. The second is passive incontinence where the person has a lack of awareness of the need to defecate before the incontinent episode.

Fecal incontinence can arise from a number of sources including, but not limited to, damage to the anal sphincters, neurologic causes, decreased distensibility of the rectum, fecal impaction, diarrhea, pregnancy, and child birth.

Current methods of treatment include medical therapy, biofeedback, sacral nerve stimulation, anal electrical stimulation, injectable bulking agent, surgery, or colostomy. The medical therapy can include bulking substances, medications that reduce stool frequency, anticholinergic medications, treatment of impaction, and defecation programs.

Several factors surrounding pregnancy and labor can contribute to an increased risk of fecal incontinence. Some of these factors can be managed, such a birth position. However, some of these factors, such as weight of the baby, cannot be managed or controlled in a way to reduce the risk of fecal incontinence. As such, a dependable method of treatment is desired for the field of pregnancy and labor relating to fecal incontinence.

Although various methods of treatment exist, these methods focus on relief rather than repair and are laborious, extensive, and costly. To overcome limitations of the aforementioned methods, a method for treatment that also serves as a method of repair of the sphincter muscles is needed without providing undue cost and burden on the patient.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a treatment probe and method for treating fecal incontinence. One embodiment of the treatment probe may include a straight, rounded treatment tip designed to accommodate rectal anatomy, an electrode assembly coupled to the treatment tip, wherein the electrode coupled to the treatment tip is configured to transfer radiofrequency energy to specific rectal structures, a temperature measuring feature coupled to the electrode assembly, wherein the temperature measuring feature coupled to the electrode assembly is configured to monitor and regulate electrode and skin temperature, a radiofrequency handle configured to connect to the treatment tip, a connector configured to connect the radiofrequency handle to a radiofrequency generator, and a protective apparatus configured to protect the radiofrequency handle.

The inventors have learned that radio frequency stimulation of rectal nerves can encourage healing of nerves, increasing their sensitivity and encouraging new nerve growth. Such stimulation also has a tightening effect on mucosal tissue to narrow the diameter of the treated canal area, and causes the healing of tissue and may help heal damaged muscle tissue and improve contractility and coordination of movement.

In another embodiment, the treatment probe may include a straight, rounded disposable treatment tip designed to accommodate rectal anatomy, an electrode assembly coupled to the treatment tip, wherein the electrode coupled to the treatment tip is configured to transfer radiofrequency energy to specific rectal structures, and wherein the electrode assembly is configured to be positioned around a full circumference of the treatment tip, a temperature measuring feature coupled to the electrode assembly, wherein the temperature measuring feature comprises a thermocouple and thermister, coupled to the electrode assembly is configured to monitor and regulate electrode and skin temperature, a reusable radiofrequency handle configured to connect to the treatment tip, a disposable protective apparatus configured to protect the radiofrequency handle, a connector configured to connect the radiofrequency handle to a radiofrequency generator source such that the treatment tip is excited by a frequency, a power level, and pattern configured to operate within a rectal cavity for the purpose of treating fecal incontinence.

In yet another embodiment of the invention, a method of treating fecal incontinence with a treatment probe may include the steps of connecting the radiofrequency handle to the radiofrequency generator, connecting the radiofrequency handle to the treatment tip, inserting the treatment tip into a rectum to treat rectal nerves, rectal muscle tissue, mucosal tissues, and facial tissues, moving the treatment tip along the rectal nerves, rectal muscle tissue, mucosal tissues, and facial tissues, and generating power with the radiofrequency generator, wherein the power creates an electric field strength that is configured to heal the rectal nerves, heal the rectal muscle tissue, tighten the mucosal tissues, and tighten the facial tissues.

The treatment probe is not limited to the shapes discussed above—another potential shape includes a gentle curved probe with an electrode on one side or alternatively on all sides wrapping around.

### BRIEF DESCRIPTION OF THE DRAWINGS

FIG. 1 is an exploded view of a device according to one embodiment of the present invention.

FIG. 2 shows the control scheme of the invention as currently embodied.

FIG. 3A and FIG. 3B are top and side views of an embodiment of disposable Interior Portion 1 from FIG. 1.

FIG. 3C shows a possible Interior Portion 1 constructed for anal usage.

FIG. 4 shows a section of vaginal wall for explanatory purposes.

FIG. 5 is a diagram of a distribution of radiofrequency induced heat according to an embodiment of the present invention.

FIG. 6 is a diagram of the front panel of a radiofrequency generator according to an embodiment of the present invention.

FIG. 7 is a diagram of a system for treating vaginal laxity according to an embodiment of the present invention.

FIG. 8 is a schematic of a radiofrequency generator and associated components according to an embodiment of the present invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

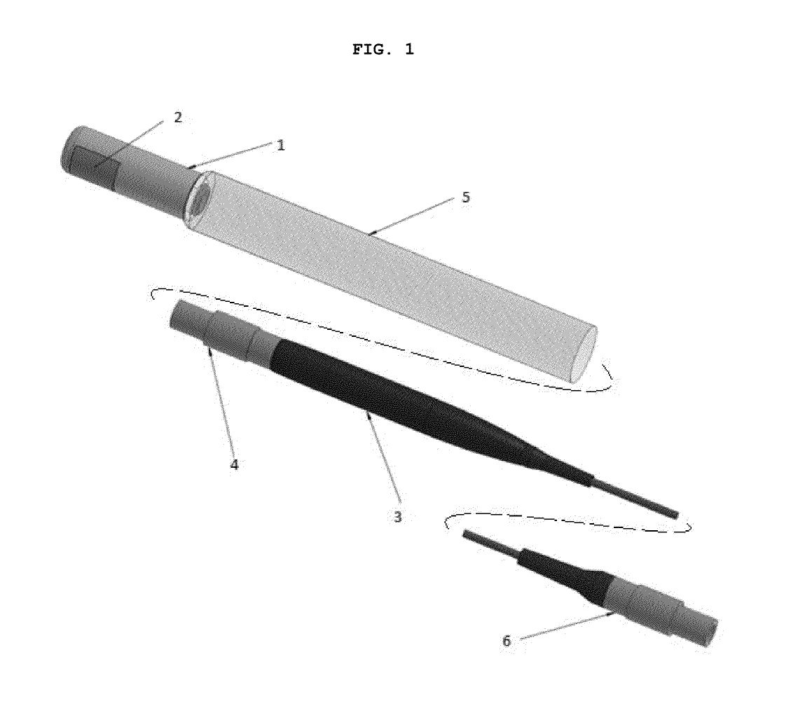

Referring now to FIG. 1, a device, according to an embodiment of the present invention, is now described in detail. The device may comprise an Interior Portion 1 (potentially disposable), a Radiofrequency (hereinafter referred to as “RF”) Electrode Assembly 3, and an RF Source 6. The Interior Portion 1 may comprise an active Treatment Electrode 2 and a Connecter 5. The Electrode Assembly 3 may be coupled to the Interior Portion 1 by the Connector 5. The Electrode Assembly may further be coupled to an RF generator by the RF Source 6.

In one embodiment of the invention, the device may comprise a non-invasive, transmucosal treatment probe as the Interior Portion 1. The transmucosal treatment probe may be configured to elevate mucosal tissue temperatures to a range approximately between 40-45° C. for the purpose of promoting tissue contracture, as well as nerve healing and improvement in sensitivity and muscle healing effects to improve strength and coordination of muscle movement. Additionally, real-time temperature monitoring may be carried out using a thermocouple. In one embodiment, the thermocouple may be integrated with a thermistor treatment probe.

In yet another embodiment of the invention, the treatment probe may be rectally inserted. The treatment tip may be applied to nerves and muscle tissue in and surrounding a rectal region. The treatment tip may then heal the nerves, heal the muscles, and tighten mucosal and facial tissues. The healed region may provide better sampling of fecal composition. The better sampling of fecal composition may allow the nerves and muscles to better determine from liquid, solid, or gas and apply a requisite amount of pressure resulting in decreased fecal incontinence. Additionally, the treatment tip may heal sphincter muscles allowing the muscles to contract more effectively and contributing further to the decreased fecal incontinence.

One of ordinary skill in the art will appreciate that the method of treatment described for vaginal walls may be applied to the rectal region to treat fecal incontinence. As such, any mention of treatment of the vaginal walls throughout this specification may be understood to be equally applied to the treatment of the rectal region in relation to treating fecal incontinence and anal fissures.

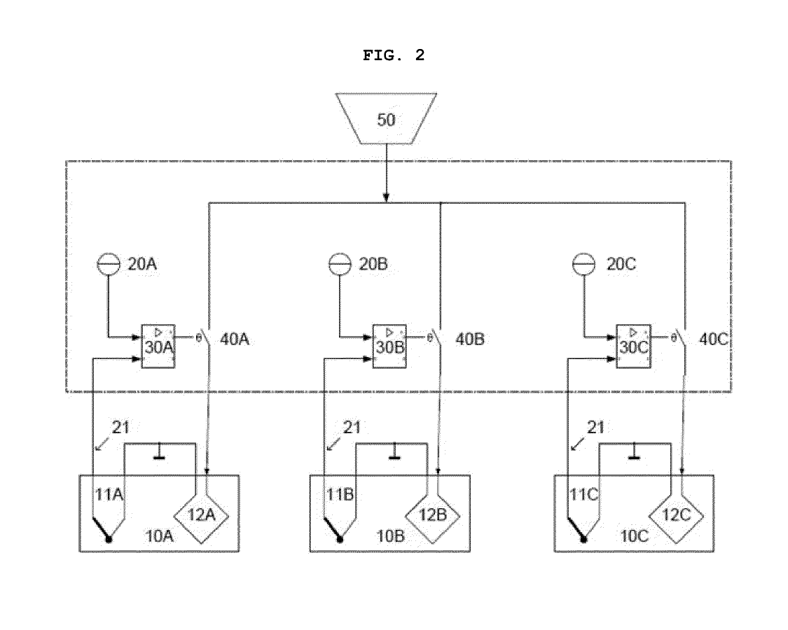

Referring now to FIG. 2, a control scheme, according to an embodiment of the present invention, is now described in detail. The control scheme may maintain an electrode set temperature during a treatment phase.

As an illustration of how a circuit functions, the disclosed embodiment may comprise a first Electrode 10A, a second Electrode 10B, and a third Electrode 10C. It should be understood that this embodiment permits temperature control of a plurality of electrodes. In other embodiments, there may be two, three, four, or more different electrodes controlled by the control scheme.

As seen in FIG. 2, each Electrode 10A 10B 10C (hereinafter referred to as “10”) may include an incorporated Temperature Sensor 11A 11B 11C (hereinafter referred to as “11”). In one embodiment of the present invention, the Temperature Sensor 11 may report the temperature at an electrode tip. Furthermore, the Temperature Sensor 11 may apply high-frequency energy to the Electrodes 10. In one embodiment, the high frequency energy may be applied using an industry available RF Tip 12A 12B 12C (hereinafter referred to as “12”). Temperature may be reported through Sensor Lines 21 from each Electrode 10 to each Control Unit 30A 30B 30C (hereinafter referred to as “30”). Each Control Unit 30 may also comprise an Input Set Temperature 20A 20B 20C (hereinafter referred to as “20”). In one embodiment, the Control Unit 30 may compare the Input Set Temperature 20 to a temperature reported by the Temperature Sensor 11. In this embodiment, the Control Unit 30 may be configured to further determine whether to open or close a Switch 40A 40B 40C (hereinafter referred to as “40”). In the current embodiment, the Switch 40 is electrical. However, those of ordinary skill in the art understand that the Switch 40 is not limited to an electrical switch. The Switch 40 may comprise a mechanical, optical, or any number of other control mechanism known in the industry.

An RF Energy Input 50 may be connected and disconnected to each Electrode 10 through the Switch 40. The connection through the Switch 40 may be operated by the Control Unit 30. In the present embodiment, the operation of the Switch 40 by the Control Unit 30 may be achieved by comparing the Input Set Temperature 20 to a temperature reported at the Temperature Sensor 11. The Control Unit 30 may be configured to deliver additional RF energy from the RF Energy Input 50 as a result of the comparison between the Input Set Temperature 20 to the temperature reported by the Temperature Sensor 11. In one embodiment, the Control Unit 30 may be configured to deliver additional RF energy so that the temperature at the Temperature Sensor 11 is approximately the Input Set Temperature 20. One of ordinary skill in the art will recognize that there are various algorithms for feedback circuits and each may be used by the Control Unit 30 to accomplish substantially the same result described in the embodiment above. One of ordinary skill in the art will also recognize that a plurality of methods exist to provide energy through heat. In another embodiment of the present invention, the RF energy may be replaced with ultrasound, heated water, laser heating, and other methods of providing energy through heat.

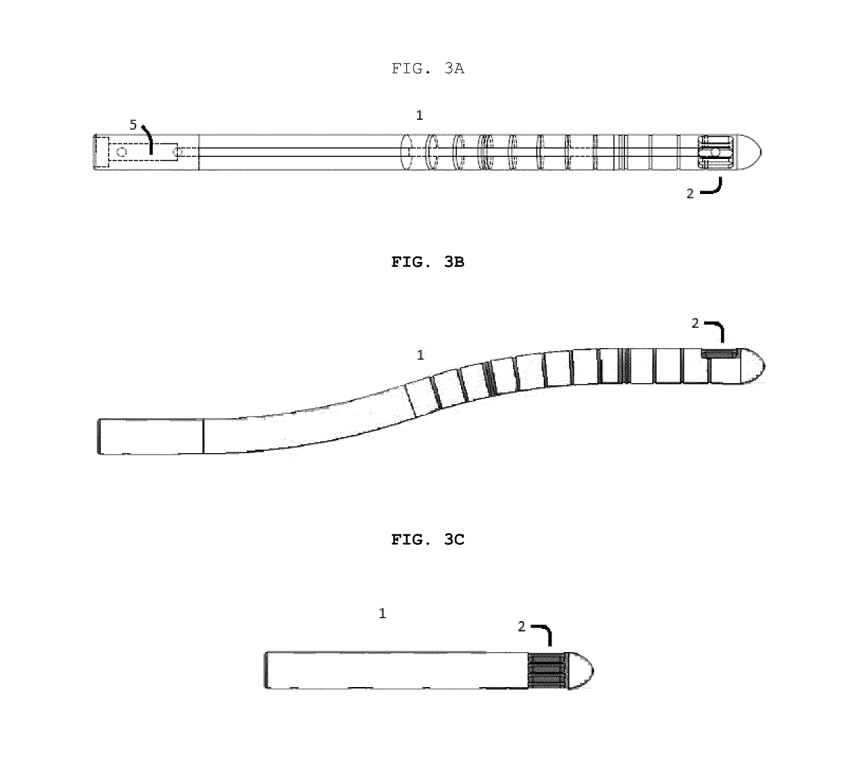

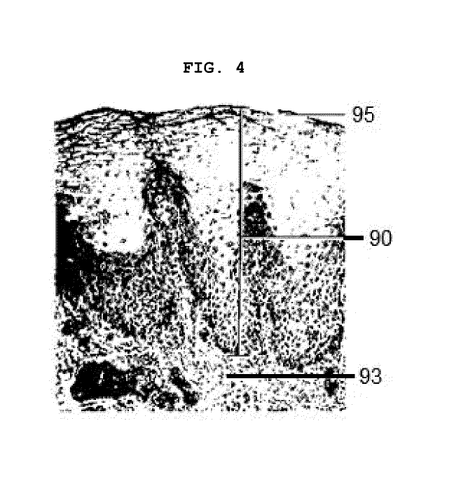

Referring now to FIG. 3A and FIG. 3B, a disposable interior portion of the device discussed in FIG. 1, according to an embodiment of the present invention, is described in detail. The disposable Interior Portion 1 of FIGS. 3A and 3B is the Interior Portion 1 of the device of FIG. 1. The Interior Portion 1 comprises an active Treatment Electrode 2 and a Connector 5. The Interior Portion 1 may further comprise a treatment tip. In one embodiment of the present invention, the active Treatment Electrode 2 may be a ThermiVa Electrode. In one embodiment of the present invention, the treatment tip may be a ThermiVa Tip. In yet another embodiment, RF energy may be delivered to the Interior Portion 1 via handheld treatment probe.

In one preferred embodiment of the present invention, the Interior Portion 1 may be curved to shape like the letter “S”. The curved Interior Portion 1 may follow natural vaginal curves. The curved Interior Portion 1 may provide a comfortable shape to a user. Additionally, in one embodiment of the present invention, the handheld treatment probe and the Interior Portion 1 may comprise a larger treatment surface. The larger surface may extend up to the entire device generating at least one of RF heat and ultrasound heat into all surfaces of the vaginal canal.

In one embodiment of the present invention, treatment of all surfaces of the vaginal canal, including, but not limited to, the labia majora and labia minora may result in reduction of labial laxity. The RF heat, as well as the other heat sources, may result in tissue coagulation and tightening and encouragement of new collagen to form. Additionally, in another embodiment of the present invention, treatment atrophic vaginitis may be possible due to collagen stimulation.

The RF heat can cause increased bloodflow, resulting in a larger amount of transudate to lubricate the vaginal canal and increasing the neurotransmitters at the nerve endings to then increase the local arteriole diameter resulting in increased bloodflow and additional transudate increase.

In another embodiment of the present invention, collagen effects on healing may result in reducing pelvic pain and vaginismus. In yet another embodiment of the present invention, at least one of tightening of periurethral tissues and pubocervical fascia, a key to improving the coordination of muscle contractions, and stronger muscle contractions of both periurethral muscles and urethral muscles to reduce stress incontinence.

In another embodiment of the present invention, at least one of tightening of posterior, anterior and sidewalls of the vaginal canal and pubocervical and rectovaginal fascias may result in reduction of pelvic prolapse symptoms.

In yet another embodiment of the present invention, at least one of tightening of anal mucosa and stimulation of sphincter muscles may result in reduction of anal incontinence.

In another embodiment of the present invention, the RF heat, as well as other types of energy through heat, may result in decreased orgasmic dysfunction.

The application of the RF heat increases blood flow in the genital regions, improving arousal, sensitivity and orgasmic response. RF heat treatments improve blood flow, as Viagra does for men. The sensitivity of nerves is positively affected, and the time to reach orgasm has been reduced by an average of 50% in the inventors' study patients. Anorgasmic patients have become orgasmic with RF heat treatments.

One of ordinary skill in the art can recognize that the above are just some potential examples of various embodiments of the use of heat through energy. One of ordinary skill in the art can also recognize that many more treatments may benefit from the application of energy through heat as applied by the device discussed hereinabove.

Referring now to FIG. 3C a disposable interior portion of the device discussed in FIG. 1, according to an embodiment of the present invention, is described in detail. The embodiment presented in FIG. 3C may represent an embodiment of the present invention configured to be used in the rectal region as opposed to the vaginal region that may be served by FIG. 3A and FIG. 3B.

The Treatment Electrode 2 of FIG. 3C may be configured to cover the full circumference of the Interior Portion 1 of the treatment probe. In another embodiment of the present invention, the Treatment Electrode 2 of FIG. 3C may be configured to cover on a portion of the circumference of the Interior Portion 1 in a same manner as the Treatment Electrode 2 of FIG. 3A and FIG. 3B.

The Interior Portion 1 of FIG. 3C may be configured to be straight with a rounded tip so as to better configure to the shape of the rectal entry and anal canal than the curved Inner Portion 1 of FIG. 3A and FIG. 3B. Additionally, the Interior Portion 1 can be constructed to be flexible and allow the device to fit patients' canals, rather than having a fixed curvature.

In one embodiment of the present invention, the Inner Portion 1 may be applied to the rectal entry and anal canal. Gradual increased heat may be applied to a lubricated rectal entry and mucosa via the Treatment Electrode 2 in a deliberate, slow, in and out, and circular motion up to approximately 3 to 5 centimeters into the anal canal. Treatment may start at approximately 35 degrees and may be gradually placed to approximately 40 to 45 degrees and kept at a maintained temperature for approximately three to five minutes or to a patient's tolerance.

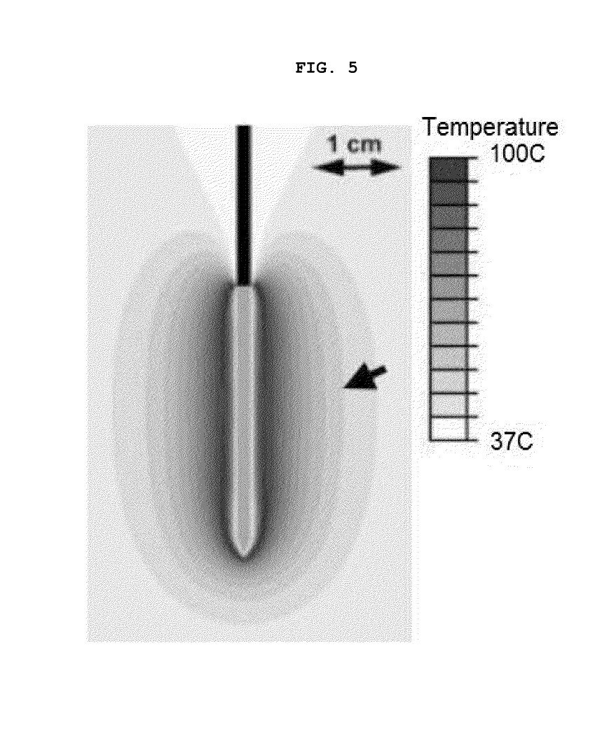

Referring to FIG. 5, a distribution of RF-induced heat, according to an embodiment of the present invention, is now described in detail. Transcutaneous RF may provide a treatment for tissue contracture. Heat from a transcutaneous temperature controlled radiofrequency device (hereinafter referred to as “TTCRFD”) may promote neo-collagenesis, denaturation of collagen cross-links, activation of wound healing pathways, contraction of collagen, and increasing in collagen fibril size.

The heat may be the result of RF experience impedance as current traverses a tissue bed. As electric current permeates a tissue layer, ions found within that tissue layer may deliver the electric current. As a result, there may an increase in kinetic activity of the ions. Increased ion kinetics and oscillations may engender resistive tissue thermogenesis. Thermogenesis may be calculable via the Specific Absorption Rate (hereinafter referred to as “SAR”) equation. SAR assesses local electrical conductivity and magnitude of local electric current density generated around an electrode.

A therapeutic benefit of the heat may be localized thermogenesis. An electric field strength generated by the RF energy may be capable of heating tissue in close proximity to the electrode. Thermal conduction may attenuate the heating of the tissue as the electrode is moved from the tissue. With proper power controls, a generated ideal thermal endpoint may occur close to the electrode. As a result, only the desired specific tissue may be affected.

The inventors have used similar techniques for ultrasound and other forms of energy, though RF energy appears to function best in practice.

Regulation of tissue temperature may derive from power control. Power in an embodiment of the present invention may be electrical voltage delivered to an RF electrode. Depending on specific tissue impedance, the power may need to be adjusted to ensure that a proper voltage is delivered to satisfy the specific tissue impedance.

Thermal sensors, for example, but not necessarily limited to, may comprise at least one of thermocouples and thermistors. The thermal sensors may be integrated within the RF electrode to adjust power to maintain a desired therapeutic temperature. In one embodiment, a thermal camera may provide real-time skin temperature monitoring. With proper controls in place, selective thermogenesis may serve as a viable treatment for numerous medical conditions.

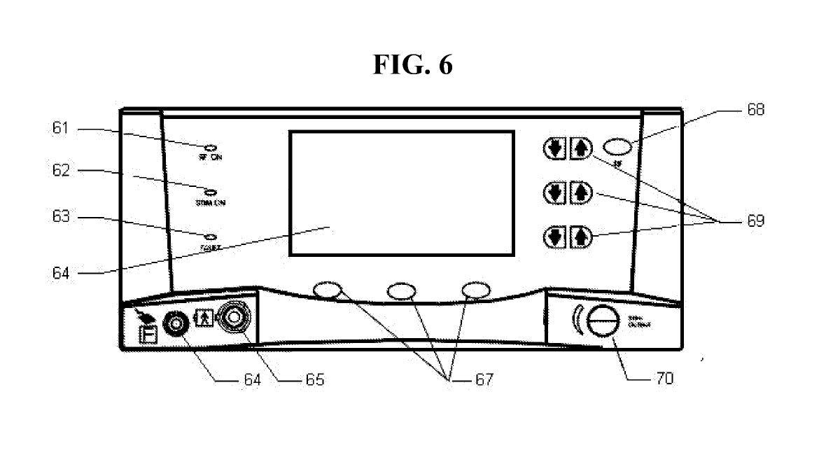

Referring to FIG. 6, an RF Source 6 or RF generator, according to an embodiment of the present invention, is now described in detail. This document will use ‘RF Generator’ and ‘RF Source’ interchangeably, as the RF generator of FIG. 6 is one embodiment of the RF Source of FIG. 1.

The RF Source 6 may comprise an RF On Light 61, a STIM On Light 62, a Fault Light 63, a Neutral Electrode Connection Port 64, a Device Connection Port 65, a Display Window 66, a plurality of Soft Keys 67, an RF On Button 68, a plurality of Up/Down Buttons 69, and a Stim Output Knob 70.

The RF On Light 61 may illuminate when the RF Generator 6 is delivering RF power. The Stim On Light 62 may illuminate when the RG generator is delivering stimulate power. The Fault Light 63 may illuminate when a fault condition is detected. The Neutral Electrode Connection Port 64 may be used to connect a neutral electrode to the RF generator. The Device Connection Port 65 may be used to connect devices to the RF Generator 6. The devices may comprise ThermiAesthetics devices among other devices.

The Display Window 66 may display a plurality of information that may comprise, but is not limited to, RF generator information, modes of operation, and operating parameters. The plurality of Soft Keys 67 may comprise, but are not limited to, STIM: Motor, ThermiTight, ThermiSmooth, ThermiRase, Help, Exit, Start, Reset, Ok, and other keys. The RF On Button 68 may, upon being pressed, start or stop RF power delivery. The plurality of UP/Down Buttons 69 may be used, upon being pressed, to increase function settings, among other operations. The Stim Output Knob 70 may adjust a stimulate output voltage. Additionally, the Stim Output Knob 70, when pressed and released, may turn stimulate power on and off.

In one embodiment of the present invention, the RF Source 6 referenced in FIG. 1 and shown in FIG. 6 may comprise the ThermiRF RF generator. Those skilled in the art will readily appreciate, however, that other RF generators may be configured in a way to still accomplish the many goals, features and advantages according to the present invention.

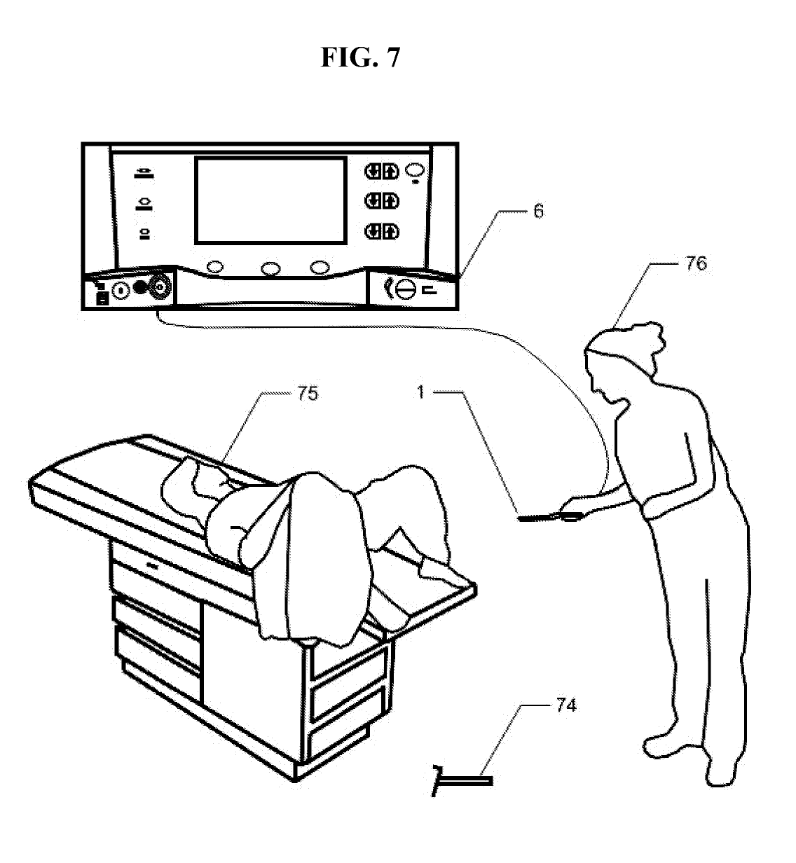

Referring to FIG. 7, a schematic of a system of treating vaginal laxity, according to an embodiment of the present invention, is now described in detail. The system comprises an RF Source 6, a Treatment Probe comprising an Interior Portion 1, Foot Pedal 74, User 75, and Provider 76.

As already noted, the RF Generator 6 is the RF Source 6 referenced in FIG. 1 and shown in FIG. 6. The Interior Portion 3 is the Interior Portion of FIG. 3A and FIG. 3B as well Interior Portion 1 of FIG. 1. The RF Generator 6 provides power and RF energy to the Interior Portion 1. The Provider 76 applies the treatment probe to the vaginal canal of the User 5. Multiple Appointments 7 may be made to repeat the procedure until desired results are achieved.

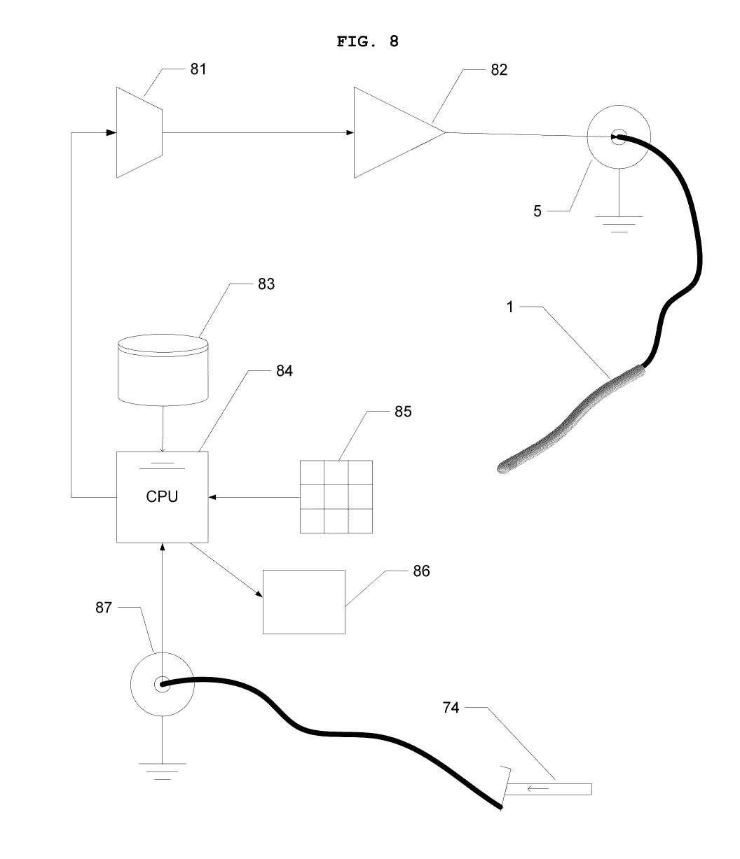

Referring to FIG. 8, a schematic of electrical components of the RF generator and associated system, according to an embodiment of the present invention, is now described in detail. In one embodiment, the RF generator may be an intelligent device comprising a Central Processing Unit (hereinafter referred to as “CPU”) 84, a Input Keys and Display 86, and an RF Oscillator 81 operated by a Foot Pedal 74.

The RF Oscillator 81 may provide energy to an Amplifier 82. The Amplifier 82 may transmit energy to a Treatment Probe 3. The Treatment Probe 3 may be electronically coupled to the RF generator by a Connector 3. The Treatment Probe 9 may provide temperature feedback to the CPU 4 through the Connector 3. A Remote Foot Pedal 8 may provide input to the CPU 4 through a Connector 7. One of ordinary skill in the art will recognize that there are numerous methods of providing input to a CPU that may each function in the place of the Foot Pedal 8.

The CPU 4 may comprise a central processing unit, with or without integrated support features, local random access storage, and Local Non-Volatile Storage 5. The Input Keys and Display 6 may be a keyboard or keypad comprising any number of keys, pads, buttons, switches, or other objects for inputting data. The Input Keys and Display 6 may be integral to the RF generator or external. The RF Oscillator 1 and Amplifier 2 may comprise functions internal, external, or both internal and external to the RF generator. Additionally, one of ordinary skill in the art can appreciate that any electronic device may comprise a remote control feature using a plurality of communications protocols.

The RF generator may also comprise a variety of computer readable media. Computer readable media can be any available media that can be accessed by a computer and includes both volatile and nonvolatile media, removable and non-removable media. By way of example, and not limitation, computer readable media may include computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, FLASH memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by a computer 610. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared and other wireless media. Combinations of any of the above should also be included within the scope of computer readable media.

Some of the illustrative aspects of the present invention may be advantageous in solving the problems herein described and other problems not discussed which are discoverable by a skilled artisan. While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

A legend of the components discussed in the application and shown on the drawings is as follows:

| | |
|---|---|
| 1 | Interior Portion |
| 2 | Treatment Electrode |
| 3 | RF Electrode Assembly |
| 6 | RF Source |
| 5 | Connector |
| 6 | RF Source |
| 10 | Electrode |
| 11 | Temperature Sensor |
| 12 | RF Tip |
| 20 | Input Set Temperature |
| 21 | Sensor Line |
| 30 | Control Unit |
| 40 | Switch |
| 50 | RF Energy Input |
| 61 | RF On Light |
| 62 | STIM On Light |
| 63 | Fault Light |
| 64 | Neutral Electrode Connection Port |
| 65 | Device Connection Port |
| 66 | Display Window |
| 67 | Soft Keys |
| 68 | RF On Button |
| 69 | Up/Down Buttons |
| 70 | Stim Output Knob |
| 74 | Foot Pedal |
| 75 | User |
| 76 | Provider |
| 81 | RF Oscillator |
| 82 | RF Amplifier |
| 83 | Computer Readable Media |
| 84 | CPU |
| 85 | Input Keys |
| 86 | Display |
| 87 | Foot Pedal Connector |
| 90 | Stratified Squamos Epithelium |
| 93 | Submucosa Tissue |
| 95 | Surface |

The inventors claim:

1. A treatment probe comprising:
a. a rounded treatment tip designed to accommodate rectal anatomy;
b. an electrode assembly coupled to the treatment tip, wherein the electrode coupled to the treatment tip is configured to transfer radiofrequency energy to specific rectal structures;
c. a temperature measuring feature coupled to the electrode assembly, wherein the temperature measuring feature coupled to the electrode assembly is configured to monitor and regulate electrode and skin temperature;
d. a radiofrequency handle configured to connect to the treatment tip;
e. a connector configured to connect the radiofrequency handle to a radiofrequency generator; and
f. a protective apparatus configured to protect the radiofrequency handle.

2. The treatment probe according to claim 1 wherein the treatment tip or treatment probe is configured to be disposable.

3. The treatment probe according to claim 1 wherein the radiofrequency handle is configured to be reusable.

4. The treatment probe according to claim 1 wherein the probe is curved or flexible and capable of changing its curvature to reflect the natural anal or vaginal canal of a patient.

5. The treatment probe according to claim 1 wherein the electrode assembly is configured to cover a full circumference of the treatment probe, the electrode assembly comprising:
a. a conductive portion;
b. a dielectric portion;
c. a radiofrequency electrode configured to capacitively couple radiofrequency energy with rectal tissue and rectal nerves when at least one of the conductive portion and the dielectric portion is in contact with a skin surface; and
d. a flex circuit electronically coupled to the radiofrequency electrode in which:
i. the conductive portion comprises a plurality of voids; and
ii. the dielectric portion is configured to be positioned between the conductive portion and the skin surface when the radiofrequency electrode is positioned at the skin surface.

6. The treatment probe according to claim 5 wherein the electrode assembly further comprises a back plate and a plurality of electrical contact pads, wherein the back plate is coupled to a support structure, and the plurality of electrical contact pads are coupled to the back plate.

7. The treatment probe according to claim 6 in which the support structure comprises a first engagement member and a second engagement member, and the first and second engagement members are configured to provide engagement and disengagement with a hand-piece support structure.

8. The treatment probe according to claim 1 wherein the temperature measuring feature is configured to be positioned along a back surface of the radiofrequency electrode.

9. The treatment probe according to claim 8 wherein the temperature measuring feature is configured to detect temperature along the back side of the radiofrequency electrode.

10. The treatment probe according to claim 1 wherein the temperate measuring feature is configured to use a thermocouple; wherein the thermocouple is integrated with a thermistor.

11. The treatment probe according to claim 10, wherein the thermistor is configured to use a feedback mechanism in which the feedback mechanism is configured to integrate a central processing unit to turn off the radiofrequency electrode when temperatures exceed a preset temperature.

12. The treatment probe according to claim 11 wherein an efficacy of the thermocouple integrated with the thermistor is configured to be most effective with manual movement of the treatment probe not exceeding one centimeter per second.

13. The treatment probe according to claim 1 wherein the protective apparatus comprises at least one of flexible latex and solid acrylic material.

14. A treatment probe comprising:
a. a rounded disposable treatment tip designed to accommodate rectal anatomy;
b. an electrode assembly coupled to the treatment tip, wherein the electrode coupled to the treatment tip is configured to transfer radiofrequency energy to specific rectal structures, and wherein the electrode assembly is configured to be positioned around a full circumference of the treatment tip;
c. a temperature measuring feature coupled to the electrode assembly, wherein the temperature measuring feature comprises a thermocouple and thermister, coupled to the electrode assembly is configured to monitor and regulate electrode and skin temperature;
d. a reusable radiofrequency handle configured to connect to the treatment tip;
e. a disposable protective apparatus configured to protect the radiofrequency handle;
f. a connector configured to connect the radiofrequency handle to a radiofrequency generator source such that the treatment tip is excited by a frequency, a power level, and pattern configured to operate within a rectal cavity for the purpose of treating fecal incontinence.

15. A method of treating fecal incontinence with a treatment probe, the treatment probe comprising a treatment tip designed to accommodate rectal anatomy, an electrode assembly coupled to the treatment tip, wherein the electrode to the treatment tip is configured to transfer radiofrequency energy to specific rectal structures, a temperature measuring feature coupled to the electrode assembly, a radiofrequency handle configured to connect to the treatment tip, a connector configured to connect the radiofrequency handle to a radiofrequency generator, and a protective apparatus configured to protect the radiofrequency handle, the method comprising the steps of:
a. connecting the radiofrequency handle to the radiofrequency generator;
b. connecting the radiofrequency handle to the treatment tip;
c. inserting the treatment tip into a rectum to treat rectal nerves, rectal muscle tissue, mucosal tissues, and facial tissues;
d. moving the treatment tip along the rectal nerves, rectal muscle tissue, mucosal tissues, and facial tissues; and
e. generating power with the radiofrequency generator, wherein the power creates an electric field strength that is configured to heal the rectal nerves, heal the rectal muscle tissue, tighten the mucosal tissues, and tighten the facial tissues.

16. The method according to claim 15 further comprising placing the treatment probe flush alongside rectal tissue to deliver the radiofrequency energy to elevate tissue temperature.

17. The method according to claim 15 furthering comprising placing the treatment probe along a rectal entry and mucosa and providing an in and out circular motion between 3 and 5 centimeters into an anal canal.

18. The method according to claim 17 further comprising positioning the treatment probe above a treatment target for a specific time to reach therapeutic temperatures without comprising skin.

19. The method according to claim 18 further comprising moving the treatment probe in a steady-paced manner from an entry angle of 35 degrees and gradually moving to 45 degrees, and wherein the specific time to reach therapeutic temperatures without comprising skin is approximately 3 to 5 minutes or to patient tolerance.

20. The method according to claim 19 wherein the treatment probe is rounded and straight to provide smooth steady-paced movement, wherein the smooth steady-paced movement is configured to provide improved patient comfort.
